(19) United States
(12) Patent Application Publication
(10) Pub. No.: US 2026/0301956 A1
(43) Pub. Date: Oct. 1, 2026

(54) Methods, Systems, and Devices for Updating a Veterinarian Radiology Worklist

(71) Applicant: IDEXX LABORATORIES, INC., Westbrook, ME (US)

(72) Inventors: Benjamin Schestopol, Scarborough, ME (US); Rebecca Baumruck, Westbrook, ME (US); Pavan Durgempudi, Portland, ME (US); Maggie Nolin, Westbrook, ME (US); Allison Spake, Westbrook, ME (US)

(21) Appl. No.: 19/629,641

(22) Filed: Mar. 26, 2026

Related U.S. Application Data
(60) Provisional application No. 63/779,163, filed on Mar. 27, 2025.

(51) Int. Cl. G16H 50/20; G16H 10/60; G16H 30/40; G16H 50/30; G16H 50/70

(52) U.S. Cl. CPC G16H 50/20; G16H 10/60; G16H 30/40; G16H 50/30; G16H 50/70

(57) ABSTRACT

A method includes receiving, by a computing device, a patient record and providing the patient record to a machine learning model. The method includes receiving, from the machine learning model, a dynamic prioritization indicator associated with the patient record. The dynamic prioritization indicator is based on the machine learning model identifying a medical condition associated with a non-human patient associated with the patient record. The method also includes comparing the dynamic prioritization indicator associated with the patient record to one or more additional dynamic prioritization indicators associated with one or more additional patient records in a veterinarian radiology worklist to determine an initial prioritization position of the patient record. The method includes, based on the comparison, generating an updated veterinarian radiology worklist. The method also includes transmitting instructions that cause the updated veterinarian radiology worklist to be displayed on a computing device.

### CROSS-REFERENCE TO RELATED APPLICATIONS

This application claims the benefit of co-pending U.S. Provisional Patent Application Serial No. 63/779,163, filed Mar. 27, 2025, which is hereby incorporated by reference in its entirety.

### FIELD OF THE DISCLOSURE

The present disclosure involves systems and methods for updating a veterinarian radiology worklist.

### BACKGROUND

Some veterinarians specialize in different fields of medicine, such as emergency veterinary medicine, surgical veterinary medicine, etc. In some scenarios, veterinarians that specialize in these fields of medicine may have to work non-traditional work hours (e.g., overnight shifts and/or weekend shifts). Veterinarians often rely on medical image interpretations to form all or part of a specific medical treatment plan and/or protocol for a non-human patient (e.g., a dog, a cat, a bird, etc.). If veterinarians rely on other medical professionals (e.g., radiologists) to interpret medical images of non-human patients, these other medical professionals may not be readily available to interpret the medical images if a request to interpret the medical images is sent during non-traditional work hours.

In these scenarios, there is a potential risk that a substantial amount of time elapses before the medical images are interpreted. To illustrate, if a veterinarian practicing in emergency veterinary medicine sends an urgent request to a radiologist to interpret medical images of a dog on a Saturday night, the radiologist may not see the urgent request until Monday morning. Furthermore, if the radiologist receives non-urgent requests between Saturday night and Monday morning, the radiologist may prioritize the non-urgent requests. For example, the non-urgent requests may be received in the form of emails that are situated at the top of the radiologist’s inbox. If the radiologist opens requests situated at the top of his or her inbox prior to opening requests that are situated at the bottom of his or her inbox, a substantial amount of time may elapse before the urgent request is processed and one or more deleterious effects may result in the medical analysis and treatment of the non-human patient.

### SUMMARY

In an example, a computer-implemented method for updating a veterinarian radiology worklist is described. In examples, the computer-implemented method includes receiving, by a computing system, a first patient record. In examples, the first patient record includes (i) one or more medical images of a first non-human patient associated with the first patient record and (ii) one or more medical indicators associated with the first non-human patient. In examples, the computer-implemented method includes providing, by the computing system, the first patient record to a machine learning model. In examples, the computer-implemented method includes receiving, by the computing system, from the machine learning model, a dynamic prioritization indicator associated with the first patient record. In examples, the dynamic prioritization indicator is based on the machine learning model identifying a medical condition associated with the first non-human patient. In examples, the identified medical condition is based on a confidence score that indicates a likelihood that the medical condition is applicable to the first non-human patient. In examples, the dynamic prioritization indicator is also based on the machine learning model generating a dynamic priority score for the first patient record based at least on the medical condition and the confidence score. In examples, the dynamic priority score indicates an urgency metric associated with the first patient record. In examples, the computer-implemented method also includes comparing, by the computing system, the dynamic prioritization indicator associated with the first patient record to one or more additional dynamic prioritization indicators associated with one or more additional patient records in the veterinarian radiology worklist to determine an initial prioritization position of the first patient record in the veterinarian radiology worklist. In examples, the computer-implemented method also includes, based on the comparison, generating, by the computing system, an updated veterinarian radiology worklist. In examples, the computer-implemented method also includes transmitting, by the computing system, instructions that cause the updated veterinarian radiology worklist to be displayed on a computing device.

In another example, a computing device is described. In examples, the computing device includes one or more processors and a tangible, non-transitory computer-readable medium comprising instructions that, when executed by the one or more processors, cause the one or more processors to perform operations. In examples, the operations include receiving a first patient record. In examples, the first patient record includes (i) one or more medical images of a first non-human patient associated with the first patient record and (ii) one or more medical indicators associated with the first non-human patient. In examples, the operations include providing the first patient record to a machine learning model. In examples, the operations include receiving a dynamic prioritization indicator associated with the first patient record. In examples, the dynamic prioritization indicator is based on the machine learning model identifying a medical condition associated with the first non-human patient. In examples, the identified medical condition is based on a confidence score that indicates a likelihood that the medical condition is applicable to the first non-human patient. In examples, the dynamic prioritization indicator is also based on the machine learning model generating a dynamic priority score for the first patient record based at least on the medical condition and the confidence score. In examples, the dynamic priority score indicates an urgency metric associated with the first patient record. In examples, the operations also include comparing the dynamic prioritization indicator associated with the first patient record to one or more additional dynamic prioritization indicators associated with one or more additional patient records in the veterinarian radiology worklist to determine an initial prioritization position of the first patient record in the veterinarian radiology worklist. In examples, the operations also include, based on the comparison, generating an updated veterinarian radiology worklist. In examples, the operations also include transmitting instructions that cause the updated veterinarian radiology worklist to be displayed on a computing device.

In another example, a tangible, non-transitory computer-readable medium is described. In examples, the tangible, non-transitory computer-readable medium includes instructions that, when executed by one or more processors, cause the one or more processors to perform operations. In examples, the operations include receiving a first patient record. In examples, the first patient record includes (i) one or more medical images of a first non-human patient associated with the first patient record and (ii) one or more medical indicators associated with the first non-human patient. In examples, the operations include providing the first patient record to a machine learning model. In examples, the operations include receiving a dynamic prioritization indicator associated with the first patient record. In examples, the dynamic prioritization indicator is based on the machine learning model identifying a medical condition associated with the first non-human patient. In examples, the identified medical condition is based on a confidence score that indicates a likelihood that the medical condition is applicable to the first non-human patient. In examples, the dynamic prioritization indicator is also based on the machine learning model generating a dynamic priority score for the first patient record based at least on the medical condition and the confidence score. In examples, the dynamic priority score indicates an urgency metric associated with the first patient record. In examples, the operations also include comparing the dynamic prioritization indicator associated with the first patient record to one or more additional dynamic prioritization indicators associated with one or more additional patient records in the veterinarian radiology worklist to determine an initial prioritization position of the first patient record in the veterinarian radiology worklist. In examples, the operations also include, based on the comparison, generating an updated veterinarian radiology worklist. In examples, the operations also include transmitting instructions that cause the updated veterinarian radiology worklist to be displayed on a computing device.

The features, functions, and advantages that have been discussed can be achieved independently in various examples or may be combined in yet other examples. Further details of the examples can be seen with reference to the following description and drawings.

### BRIEF DESCRIPTION OF THE FIGURES

The above, as well as additional features will be better understood through the following illustrative and non-limiting detailed description of example embodiments, with reference to the appended drawings.

FIG. 1 illustrates a diagram of an example system for updating a veterinarian radiology worklist, according to one or more embodiments.

FIG. 2 illustrates a diagram of an example system for reprioritizing a veterinarian radiology worklist, according to one or more embodiments.

FIG. 3 illustrates a diagram of an example system for classifying patient records in a veterinarian radiology worklist, according to one or more embodiments.

FIG. 4 illustrates a diagram of another example system for updating a veterinarian radiology worklist, according to one or more embodiments.

FIG. 5 illustrates a diagram of an example process for training a machine learning model to update a veterinarian radiology worklist, according to one or more embodiments.

FIG. 6 illustrates a method, according to one or more embodiments.

All the figures are schematic, not necessarily to scale, and generally only show parts which are necessary to elucidate example embodiments, wherein other parts may be omitted or merely suggested.

### DETAILED DESCRIPTION

Example embodiments will now be described more fully hereinafter with reference to the accompanying drawings. That which is encompassed by the claims may, however, be embodied in many different forms and should not be construed as limited to the embodiments set forth herein; rather, these embodiments are provided by way of example. Furthermore, like numbers refer to the same or similar elements or components throughout.

Within examples, the disclosure is directed to dynamically updating a veterinarian radiology worklist using machine learning to prioritize interpretation of particular medical images. To illustrate, a non-human patient (e.g., a pet or animal) may undergo medical imaging (e.g., x-ray imaging, gamma ray imaging, computed tomography scanning, etc.) and a resulting set of radiology images may be generated. In the form of a patient record, a veterinarian may send the radiology images to a specialist (e.g., a radiologist) for interpretation to diagnose a medical condition of the non-human patient. The patient record can also include medical indicators associated with the non-human patient, such as an age, a weight, a species, a breed, one or more symptoms, a preexisting condition, and/or a geographical location of the non-human patient. Because the radiologist may receive numerous patient records, the techniques described herein utilize machine learning to dynamically prioritize patient records in a veterinarian radiology worklist that is accessible to the radiologist. Higher priority patient records may be at the top of the veterinarian radiology worklist so that the radiologist can work on (e.g., interpret) the associated radiology images prior to interpreting radiology images associated with lower priority patient records.

To generate and/or update the veterinarian radiology worklist, in examples, the patient record is provided to a machine learning model. For example, based on the radiology images and the medical indicators, the machine learning model may identify a medical condition of the non-human patient and a confidence score associated with the medical condition. Based on the medical condition, the machine learning model may determine an urgency metric, indicating an urgency of the medical condition. Urgent medical conditions, such as traumatic injuries, may have a higher priority than non-urgent medical conditions, such as fleas. The machine learning model may generate a dynamic prioritization indicator for the patient record based on the urgency metric.

The dynamic prioritization indicator for the patient record may be compared to dynamic prioritization indicators of other patient records to determine which patient records have the highest priority. As described above and in greater detail below, the veterinarian radiology worklist may be updated by inserting the patient record according to its priority with respect to other patient records in the veterinarian radiology worklist. As described below, once a patient record is inserted into the veterinarian radiology worklist according to its initial priority, the priority of the patient record can dynamically change as time elapses and no action is performed. For example, after a particular period of time, a non-urgent patient record may be reclassified as urgent or critical and may be assigned a higher priority in the veterinarian radiology worklist.

Accordingly, features of the present disclosure can help to address these and other issues to provide an improvement to select technical fields. More specifically, features of the present disclosure help address issues within and provide improvements for select technical fields, which include for example, veterinary and medical care communication systems, medical record and medical treatment protocol and triage processing systems, computer-based analysis systems, and graphical user interfaces (GUIs), all of which also help further improve medical treatments for one or more non-human patients. These features and more will now be described

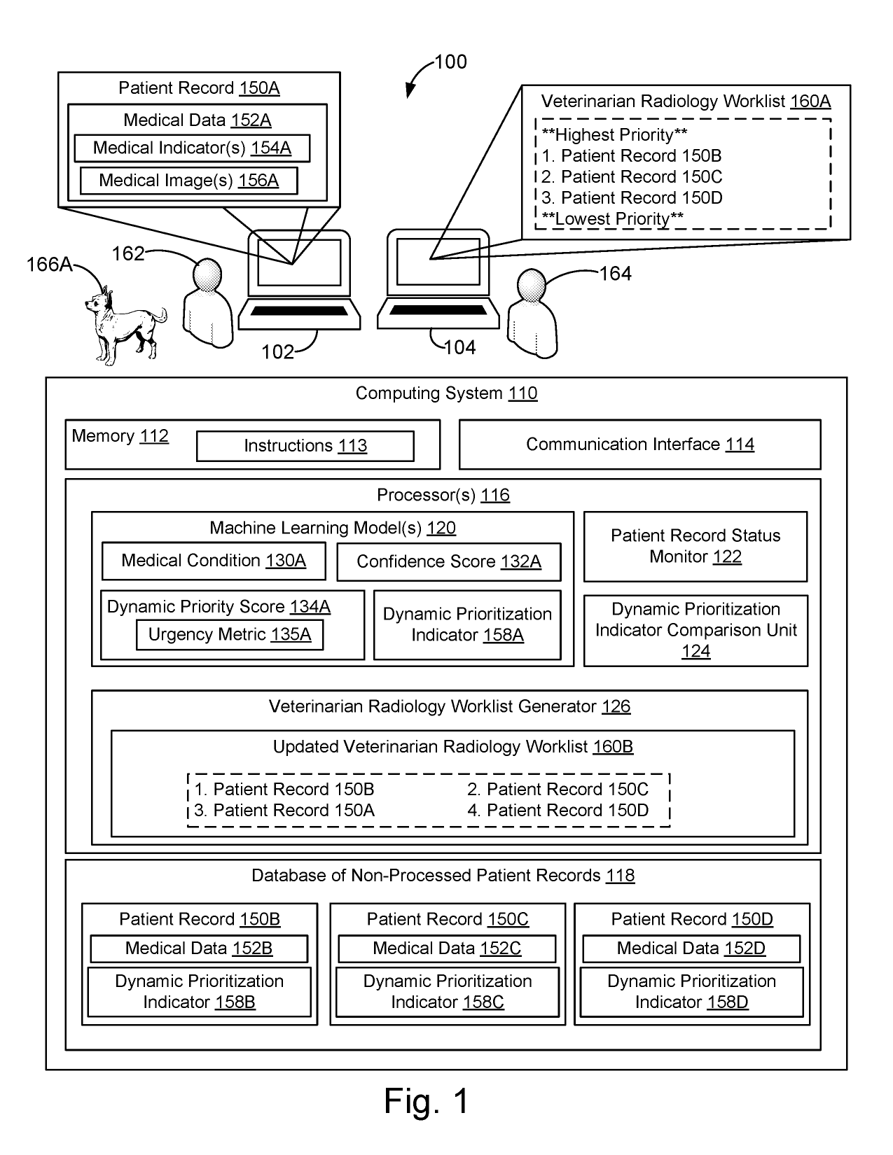

Particular implementations are described herein with reference to the figures. In the description, common features may be designated by common reference numbers throughout the figures. In some figures, multiple instances of a particular type of feature are used. Although these features are physically and/or logically distinct, the same reference number is used for each, and the different instances are distinguished by addition of a letter to the reference number. When the features as a group or a type are referred to herein (e.g., when no particular one of the features is being referenced), the reference number is used without a distinguishing letter. However, when one particular feature of multiple features of the same type is referred to herein, the reference number is used with the distinguishing letter. For example, referring to FIG. 1, patient records are illustrated and associated with reference numbers 150A, 150B, 150C, and 150D. When referring to a particular one of the patient records, such as the patient record 150A, the distinguishing letter “A” is used. However, when referring to any one of the patient records or to the patient records as a group, the reference number 150 may be used without a distinguishing letter.

Referring now to the figures, FIG. 1 is a diagram of an example system 100 for updating a veterinarian radiology worklist. The system 100 includes a computing device 102, a computing device 104, and a computing system 110. In examples, the computing devices 102, 104 may be one of laptop computers, desktop computers, personal digital assistants (PDAs), tablets, workstation terminals, mobile phones, controllers, or any other devices that can receive, transmit, and display information, such as medical records. In one scenario, the computing device 102 may be accessible to a first user (e.g., a veterinarian or veterinary technician or the like) 162, and the computing device 104 may be accessible to a second user (e.g., a specialist such as a radiologist or the like) 164.

As described below, the computing system 110 may be configured to dynamically update a veterinarian radiology worklist 160 such that urgent patient records 150 (e.g., higher priority patient records 150) are at the top of the veterinarian radiology worklist 160 and less urgent patient records 150 (e.g., lower priority patient records 150) are at the bottom of the veterinarian radiology worklist 160. The second user 164 can access the veterinarian radiology worklist 160 at the computing device 104 and interpret medical images 156 (e.g., radiology images, x-ray images, gamma-ray images, etc.) associated with higher priority patient records 150 before interpreting the medical images 156 associated with the lower priority patient records 150.

In some examples, the computing system 110 may be integrated into the computing device 102. For example, the computing device 102 accessible to the first user 162 (e.g., the party requesting interpretation of the medical images 156) may dynamically update the veterinarian radiology worklist 160 and send the updated veterinarian radiology worklist 160 to the computing device 104 accessible to the second user 164 (e.g., the party interpreting the medical images 156). In some examples, the computing system 110 may be integrated into the computing device 104. For example, the computing device 104 accessible to second user 164 may dynamically update the veterinarian radiologist worklist 160 based on incoming patient records from the computing device 102 accessible to the first user 162. In some examples, the computing system 110 may be integrated into a server (not shown). For example, patient records 150 from the computing device 102 accessible to the first user 162 may be transmitted to the server, and the server may (i) dynamically update the veterinarian radiology worklist 160 based on the incoming patient records 150 and (ii) send the updated veterinarian radiology worklist 160 to the computing device 104 accessible to the second user 164.

The computing system 110 includes a memory 112, a communication interface 114, one or more processors 116 (herein referred to interchangeably as “the processor(s) 116” or the processor 116), and a database 118 of non-processed patient records. The memory 112 can include one or more volatile, non-volatile, removable, and/or non-removable storage components, such as magnetic, optical, or flash storage, and/or can be integrated in whole or in part with the processor(s) 116. Further, the memory 112 can take the form of a non-transitory computer-readable storage medium, having stored thereon program instructions 113 (e.g., compiled or non-compiled program logic and/or machine code) that, when executed by the processor(s) 116, cause the computing system 110 to perform one or more acts and/or functions, such as those described in this disclosure. As such, the computing system 110 can be configured to perform one or more acts and/or functions, such as those described in this disclosure. Such program instructions 113 can define and/or be part of a discrete software application. In some instances, the computing system 110 can execute program instructions 113 in response to receiving an input (e.g., a patient record 150) from the communication interface 114. The memory 112 can also store other types of data, such as those types described in this disclosure.

The communication interface 114 can allow the computing system 110 to connect to and/or communicate with other entities, such as the computing devices 102, 104, according to one or more protocols. In one example, the communication interface 114 can be a wired interface, such as an Ethernet interface or a high-definition serial-digital-interface (HD-SDI). In another example, the communication interface 114 can be a wireless interface, such as a cellular or WI FI interface. In this disclosure, a connection can be a direct connection or an indirect connection, the latter being a connection that passes through and/or traverses one or more entities, such as such as a router, switcher, or other network device. Likewise, in this disclosure, a transmission can be a direct transmission or an indirect transmission.

The processor 116 can include a general-purpose processor (e.g., a microprocessor) and/or a special-purpose processor (e.g., a digital signal processor (DSP)). The processor 116 includes one or more machine learning models 120 (herein referred to as “the machine learning model 120”), a patient record status monitor 122, a dynamic prioritization indicator comparison unit 124, and a veterinarian radiology worklist generator 126. In some examples, one or more components of the processor 116 may be implemented using dedicated hardware. To illustrate, in some examples, one or more components of the processor 116 may be implemented via an application-specific integrated circuit (ASIC) or a field-programmable gate array (FPGA) device. In some examples, one or more components of the processor 116 may be implemented using software or firmware. To illustrate, in some examples, operations performed by one or more of the components of the processor 116 can be implemented by executing the instructions 113 stored in the memory 112.

As referenced above, the computing system 110 may be configured to dynamically update a veterinarian radiology worklist 160 such that urgent patient records 150 (e.g., higher priority patient records 150) are at the top of the veterinarian radiology worklist 160 and less urgent patient records 150 (e.g., lower priority patient records 150) are at the bottom of the veterinarian radiology worklist 160. To illustrate, a veterinarian radiology worklist 160A is accessible via the computing device 104. The veterinarian radiology worklist 160A ranks patient records 150B, 150C, 150D from highest priority to lowest priority. In the illustrative example of FIG. 1, in the veterinarian radiology worklist 160A, the patient record 150B has the highest priority, the patient record 150C has the next highest priority, and the patient record 150D has the lowest priority. Thus, according to the veterinarian radiology worklist 160A, the second user 164 associated with the computing device 104 is encouraged to interpret medical images 156 associated with the patient record 150B prior to interpreting medical images 156 associated with the other patient records 150C, 150D.

The computing system 110 may be configured to receive, via the communication interface 114, a patient record 150A from the computing device 102. For example, the first user 162 associated with the computing device 102 may send the patient record 150A to the computing system 110. The patient record 150A may include medical data 152A of a non-human patient 166A (e.g., a cat, a dog, a bird, etc.). For example, the patient record 150A may include one or more medical indicators 154A associated with the non-human patient 166A and one or more medical images 156 associated with the non-human patient 166A. The medical indicators 154A may include information stored in association with the one or more medical records associated with the non-human patient 166A. As non-limiting examples, the one or more medical indicators may include an age of the non-human patient 166A, a weight of the non-human patient 166A, a species of the non-human patient 166A, a breed of the non-human patient 166A, one or more symptoms of the non-human patient 166A, a preexisting condition of the non-human patient 166A, a geographical location of the non-human patient 166A, or a combination thereof.

In some examples, the one or more medical indicators 154A associated with the non-human patient 166A includes information stored in association with one or more medical records associated with another non-human patient 166 that shares one or more attributes with the non-human patient 166A. The one or more attributes may include an age, a weight, a species, a breed, one or more symptoms, a preexisting condition, a geographical location, or a combination thereof.

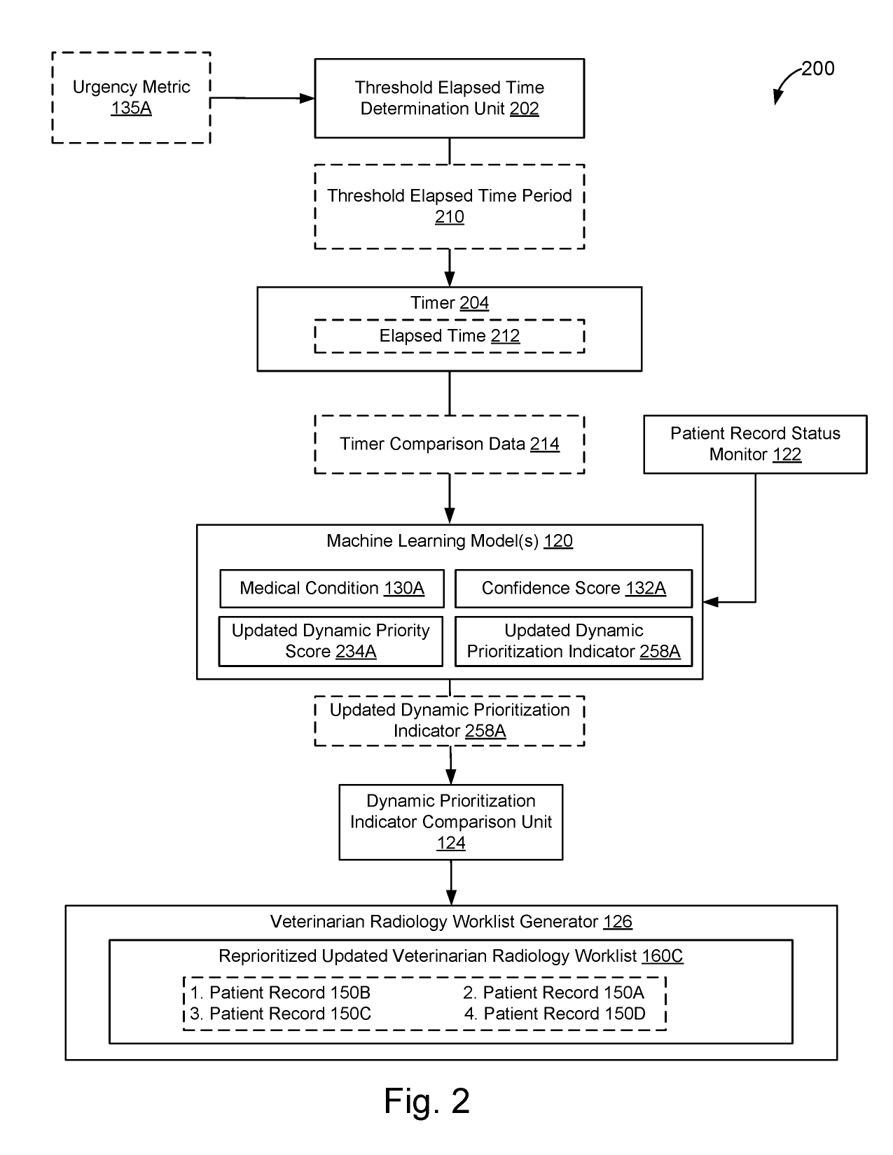

The computing system 110 (e.g., the processor 116) may be configured to provide the patient record 150A to the machine learning model 120. Based on the patient record 150A, the machine learning model 120 may be configured to identify a medical condition 130A associated with the non-human patient 166A. The identified medical condition 130A may be based on a confidence score 132A that indicates a likelihood that the medical condition 130A is applicable to the non-human patient 166A. The machine learning model 120 may also be configured to generate a dynamic priority score 134A for the patient record 150A based at least on the medical condition 130A and the confidence score 132A. The dynamic priority score 134A may indicate an urgency metric 135A associated with the patient record 150A. For example, if the medical condition 130A is associated with a serious or life-threatening condition (e.g., brain injury, traumatic injury, etc.), the urgency metric 135A, and thus the dynamic priority score 134A, may be relatively high. However, if the medical condition 130A is associated with a non-serious condition (e.g., fleas), the urgency metric 135A, and thus the dynamic priority score 134A, may be relatively low. As described with respect to FIG. 2, the urgency metric 135A may be used to determine when and how to reprioritize patient records 150 in the veterinarian radiology worklist 160.

In some examples, the determination of the dynamic priority score 134A by the machine learning model 120 may further be based on a medical provider associated with the non-human patient 166A. For example, if the first user 162 is a preferred medical provider, the dynamic priority score 134A may be higher than if the first user 162 is not a preferred medical provider (e.g., an “out-of-network” medical provider).

Based on the medical condition 130A, the confidence score 132A, and the dynamic priority score 134A, the machine learning model 120 may be configured to generate a dynamic prioritization indicator 158A associated with the patient record 150A. The dynamic prioritization indicator 158A associated with the patient record 150A is a metric that is usable to determine where the patient record 150A should be positioned (e.g., a priority of the patient record 150A) on the veterinarian radiology worklist 160. In some examples, urgent medical conditions 130 with high dynamic priority scores 134 may result in higher dynamic prioritization indicators 158 that indicate the relevant patient records 150 should have higher priority on the veterinarian radiology worklist 160.

As described above, although the machine learning model 120 is depicted within the computing system 110, in some examples, the machine learning model 120 may be located at a remote server to reduce the amount of data stored at the computing system 110 and improve processing speed at the computing system 110. In these examples, the computing system 110 may transmit the patient record 150 to the machine learning model 120 at the remote server and receive the dynamic prioritization indicator 158A from the machine learning model 120 at the remote server.

The patient record status monitor 122 may be configured to monitor whether the second user 164 has processed patient records 150 in the veterinarian radiologist worklist 160A. For example, the second user 164 may send, via the computing device 104, an indication that he or she has processed a particular patient record 150 (e.g., interpreted corresponding medical images 156). Once the indication is received, the patient record status monitor 122 may remove the particular patient record 150 from the veterinarian radiology worklist 160A. Thus, at any given time, the patient record status monitor 122 may be configured to determine which patient records 150 remain in the veterinarian radiology worklist 160A and how long each patient record 150 has been in the veterinarian radiology worklist 160A.

The database 118 of non-processed patient records may be controlled by the patient record status monitor 122. For example, the database 118 of non-processed patient records stores patient records 150 that have yet to be processed by second user 164. In the illustrative example of FIG. 1, the database 118 of non-processed patient records includes the patient record 150B, the patient record 150C, and the patient record 150D. As shown in FIG. 1, the patient records 150B, 150C, 150D in the database 118 match the patient records 150B, 150C, 150D in the veterinarian radiology worklist 160A at the computing device 104. The patient record 150B may include medical data 152B and a dynamic prioritization indicator 158B for a corresponding non-human patient 166, the patient record 150C may include medical data 152C and a dynamic prioritization indicator 158C for a corresponding non-human patient 166, and the patient record 150D may include medial data 152D and a dynamic prioritization indicator 158D for a corresponding non-human patient 166.

The dynamic prioritization indicator comparison unit 124 may be configured to compare the dynamic prioritization indicator 158A associated with the patient record 150A to one or more additional dynamic prioritization indicators 158B, 158C, 158D associated with one or more additional patient records 150B, 150C, 150D in the veterinarian radiology worklist 160A to determine an initial prioritization position for the patient record 150A in the veterinarian radiology worklist 160. Based on the comparison, the veterinarian radiology worklist generator 126 may be configured to generate an updated veterinarian radiology worklist 160B. As illustrated in FIG. 1, the patient record 150A has the third highest priority in the updated veterinarian radiology worklist 160B. Thus, the dynamic prioritization indicator 158A associated with the patient record 150A indicates that the patient record 150A has a lower priority than the patient records 150B, 150C and a higher priority than the patient record 150D.

The communication interface 114 may be configured to send the patient record 150A, the medical condition 130A, and the confidence score 132A to the computing device 104. Thus, when processing the patient record 150A (e.g., interpreting the medical images 156A), the second user 164 may be privy to the medical condition 130A determined by the machine learning model 120. This information may reduce the amount of time needed for the second user 164 to interpret the medical images 156A. For example, the second user 164 may be relegated to confirming whether the medical condition 130A identified by the machine learning model 120 is accurate. In some scenarios, this determination from the second user 164 may be provided to the machine learning model 120 as feedback to further train (e.g., retrain) the machine learning model 120.

Additionally, the communication interface 114 may configured to transmit instructions that cause the updated veterinarian radiology worklist 160B to be displayed at the computing device 104 accessible to the second user 164. For example, the updated veterinarian radiology worklist 160B may be accessible to the second user 164 via a software application that is installed on the computing device 104. The computing system 110 may update data in the software application such that the veterinarian radiology worklist 160A is replaced with the updated veterinarian radiology worklist 160B. In some examples, the computing system 110 may have special access to an email account associated with the second user 164. In these examples, if the patient records 150 are received via emails directed to the email account, the computing system 110 may insert prioritization indicators in the second user’s 164 inbox to emphasize the priority of the patient records 150 in the updated veterinarian radiology worklist 160B.

The techniques described with respect to FIG. 1 reduce the likelihood that urgent patient records 150 are ignored by the second user 164 in lieu of the non-urgent patient records 150. For example, by generating and updating the veterinarian radiology worklist 160 based on the dynamic prioritization indicators 158, the second user 164 will be able to easily identify the most urgent patient records 150 can provide radiologist readings (e.g., interpretations) to those patient records 150, first.

FIG. 2 is a diagram of an example system 200 for reprioritizing a veterinarian radiology worklist.

The system 200 includes a threshold elapsed time determination unit 202, a timer 204, the machine learning model 120, the patient record status monitor 122, the dynamic prioritization indicator comparison unit 124, and the veterinarian radiology worklist generator 126. In some examples, components of the system 200 may be integrated into the computing system 110 of FIG. 1. In particular, the components of the system 200 may be integrated into the processor 116 depicted in FIG. 1.

The system 200 may be configured to dynamically generate a reprioritized updated veterinarian radiology worklist 160C based on timing factors. To illustrate, the urgency metric 135A associated with the patient record 150A may be provided to the threshold elapsed time determination unit 202. The urgency metric 135A may indicate how quickly the second user 164 should process the patient record 150A (e.g., interpret the medical images 156A). Based on the urgency metric 135A associated with the patient record 150A, the threshold elapsed time determination unit 202 may be configured to determine a threshold elapsed time period 210 for reprioritizing the patient record 150A in the updated veterinarian radiology worklist 160B. The threshold elapsed time period 210 may be provided to the timer 204.

The timer 204 may be configured to monitor an elapsed time 212 since generation of the updated veterinarian radiology worklist 160B. Based on comparison between the elapsed time 212 and the threshold elapsed time period 210, the timer 204 may generate timer comparison data 214 that indicates whether the elapsed time 212 has exceed the threshold elapsed time period 210. The timer comparison data 214 may be provided to the machine learning model 120.

The patient record status monitor 122 may determine whether an action has been performed on the patient record 150A. For example, as described with respect to FIG. 1, the patient record status monitor 122 may indicate whether the second user 164 processed the patient record 150A (e.g., interpreted the medical images 156A). The determination by the patient record status monitor 122 may also be provided to the machine learning model 120. Thus, based on the timer comparison data 214 and the information from the patient record status monitor 122, the machine learning model may be configured to determine whether an action has been performed on the patient record 150A prior to the elapsed time 212 exceeding the threshold elapsed time period 210.

In response to determining that no action has been performed on the patient record 150A prior to the elapsed time 212 exceeding the threshold elapsed time period 210, the machine learning model 120 may be configured to update the dynamic priority score 134 (to an updated dynamic priority score 234A) based on an escalated urgency metric. Based on the updated dynamic priority score 234A, the machine learning model 120 may generate an updated dynamic prioritization indicator 258A associated with the patient record 150A. The updated dynamic prioritization indicator 258A is provided to the dynamic prioritization indicator comparison unit 124.

The dynamic prioritization indicator comparison unit 124 may be configured to compare the updated dynamic prioritization indicator 258A associated with the patient record 150A to the one or more additional dynamic prioritization indicators 158B, 158C, 158D associated with the one or more additional patient records 150B, 150C, 150D to determine an updated prioritization position of the patient record 150A. Based on the updated prioritization position, the veterinarian radiology worklist generator 126 may be configured to reprioritize the patient record 150A in the updated veterinarian radiology worklist 160B to generate the reprioritized updated veterinarian radiology worklist 160C. As illustrated in FIG. 2, the patient record 150A has the second highest priority in the reprioritized updated veterinarian radiology worklist 160C. Thus, the updated dynamic prioritization indicator 258A associated with the patient record 150A indicates that the patient record 150A has a lower priority than the patient record 150B and a higher priority than the patient records 150C, 150D. Therefore, the updated prioritization position of the patient record 150A has a higher priority than the initial prioritization position of the patient record 150A.

In a similar manner as described with respect to FIG. 1, the communication interface 114 may transmit instructions that cause the reprioritized updated veterinarian radiology worklist 160C to be displayed on the computing device 104. However, in scenarios where the machine learning model 120 determine that no action has been performed on the patient record 150A prior to the elapsed time 212 exceeding the threshold elapsed time period 210, the communication interface 114 may transmit (or retransmit) instructions that cause the updated worklist to be displayed on another computing device (not shown). In some scenarios, the other computing device may be associated with another radiologist.

The techniques described with respect to FIG. 2 enable the priority of the patient record 150A to be dynamically updated based on the urgency metric 135A associated with the patient record 150A. For example, in some scenarios, the medical condition 130A may not be urgent at first, but as time elapses, the medical condition 130A can become more serious. One example of such a medical condition 130A may be an infection. If the second user 164 has not taken an action on the patient record 150A after expiration of the threshold elapsed time period 210, the techniques described with respect to FIG. 2 enable the increasingly serious medical condition 130A take higher priority.

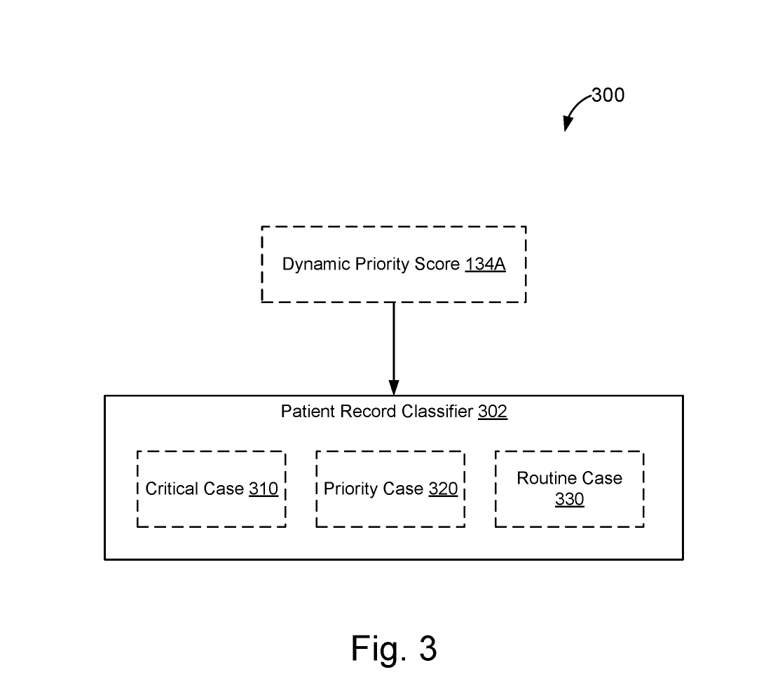

FIG. 3 is a diagram of an example system 300 for classifying patient records in a veterinarian radiology worklist. The system 300 includes a patient record classifier 302. In some examples, the patient record classifier 302 may be integrated into the computing system 110 of FIG. 1. In particular, the patient record classifier 302 may be integrated into the processor 116 depicted in FIG. 1.

The patient record classifier 302 may be configured to receive the dynamic priority score 134A associated with the patient record 150A and classify the patient record 150A based on the dynamic priority score 134A. In the illustration of FIG. 3, the patient record 150A may be classified as a critical case 310 if action should be performed on the patient record 150A before expiration of a first time period, the patient record 150A may be classified as a priority case 320 if action should be performed on the patient record 150A before expiration of a second time period, and the patient record 150A may be classified as a routine case 330 if action should be performed on the patient record 150A before expiration of a third time period. In some examples, the first time period is one hour, the second time period is four hours, and the third time period is twenty-four hours. However, it should be understood that in other examples, the duration of the time periods may differ. In some examples, the duration of the time periods is user configurable. As a non-limiting example, the first user 162 may set the duration of the time periods.

In some examples, the classification of the patient record 150A is configured to change based on the elapsed time 212 since inserting the patient record in the updated veterinarian radiology worklist 160B. For example, when the elapsed time 212 exceeds the threshold elapsed time period 210, the classification of the patient record 150A may change from a priority case 320 to a critical case 310. In some examples, the classification of the patient record 150A may be included in the veterinarian radiology worklist 160.

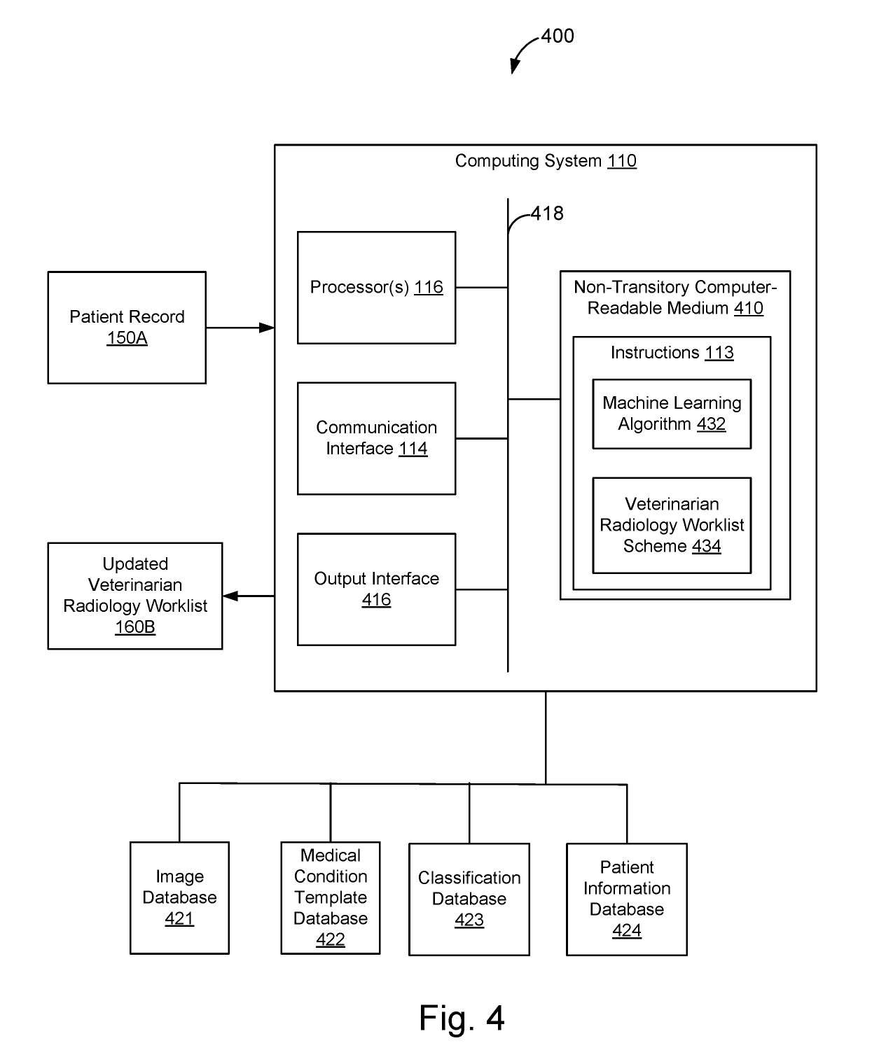

FIG. 4 is a diagram of another example system 400 for updating a veterinarian radiology worklist is depicted. The system 400 includes the computing system 110. The patient record 150A is provided to the computing system 110.

The computing system 110 includes the processor 116 and a non-transitory computer-readable medium 410 (e.g., the memory 112) storing instructions 113 executable by the processor 116 to perform functions for updating a veterinarian radiology worklist. The computing system 110 is shown as a stand-alone component in FIG. 4. However, as indicated above, the computing system 110 may be incorporated within the computing device 102 or the computing device 104.

To perform functions noted above, the computing system 110 also includes the communication interface 114, an output interface 416, and each component of the computing system 110 is connected to a communication bus 418. The computing system 110 may also include hardware to enable communication within the computing system 110 and between the computing system 110 and other devices (e.g., the computing devices 102, 104 and/or a server). The hardware may include transmitters, receivers, and antennas, for example.

The communication interface 114 may be a wireless interface and/or one or more wireline interfaces that allow for both short-range communication and long-range communication to one or more networks or to one or more remote devices. Such wireless interfaces may provide for communication under one or more wireless communication protocols, Bluetooth, Wi-Fi (e.g., an institute of electrical and electronic engineers (IEEE) 802.11 protocol), Long-Term Evolution (LTE), cellular communications, near-field communication (NFC), and/or other wireless communication protocols. Such wireline interfaces may include an Ethernet interface, a Universal Serial Bus (USB) interface, or similar interface to communicate via a wire, a twisted pair of wires, a coaxial cable, an optical link, a fiber-optic link, or other physical connection to a wireline network. Thus, the communication interface 114 may be configured to receive input data from one or more devices, and may also be configured to send output data to other devices.

The non-transitory computer readable medium 410 may include or take the form of memory (e.g., the memory 112), such as one or more computer-readable storage media that can be read or accessed by the processor 116. The non-transitory computer readable medium 410 can include volatile and/or non-volatile storage components, such as optical, magnetic, organic or other memory or disc storage, which can be integrated in whole or in part with the processor 116. In some examples, the non-transitory computer readable medium 410 can be implemented using a single physical device (e.g., one optical, magnetic, organic or other memory or disc storage unit), while in other examples, the non-transitory computer readable medium 410 can be implemented using two or more physical devices. The non-transitory computer readable medium 410 thus is a computer readable storage, and the instructions 113 are stored thereon. The instructions 113 include computer executable code.

The processor 116 may be a general-purpose processor or a special purpose processor (e.g., a digital signal processor, an ASIC, etc.). The processor 116 may receive inputs from the communication interface 114 (e.g., the patient record 150A), and process the inputs to generate outputs that are stored in the non-transitory computer readable medium 410. The processor 116 can be configured to execute the instructions 113 (e.g., computer-readable program instructions) that are stored in the non-transitory computer readable medium 410 and are executable to provide the functionality of the computing system 110 described herein.

The output interface 416 outputs information for reporting or storage (e.g., the updated veterinarian radiology worklist 160B), and thus, the output interface 416 may be similar to the communication interface 414 and can be a wireless interface (e.g., transmitter) or a wired interface as well.

The system 400 can also include or be coupled to a number of databases, such as an image database 421, a medical condition template database 222, a classification database 423, and a patient information database 424. In FIG. 4, the additional databases are shown as separate components of the computing system 110; however, each database may alternatively be integrated within the computing system 110. Access of the databases further enables the computing system 110 to perform functions as described herein. Functionality and content of the databases is described below.

Within one example, in operation, when the instructions 113 are executed by the processor 116, the processor 116 is caused to perform functions including using a machine learning algorithm 432 to determine the medical condition 130A associated with the patient record 150A, the confidence score 132A for the medical condition 130A, the dynamic priority score 134A associated with the patient record 150A, and the dynamic prioritization indicator 158A associated with the patient record 150A.

The machine learning algorithm 432 uses statistical models to determine the medical condition 130A, the confidence score 132A, the dynamic priority score 134A, and the dynamic prioritization indicator 158A effectively without using explicit instructions, but instead, can rely on patterns and inferences. In one example, the machine learning algorithm 432 accesses the image database 421, which includes previously labeled medical images that are indexed using a multi-dimensional indexing scheme based on relevant features/parameters. In such examples, the features/parameters extracted from the medical images 156A under consideration can be compared to the feature data of labeled medical images in the image database 421 to identify particular anatomy or view, and help identify the label of the image captured.

In another example, the machine learning algorithm 432 can access the medical condition template database 422, which includes templates of different medical conditions constructed using information obtained from the image database 421. For example, feature data over a plurality of known and labeled medical images can be processed using statistical techniques to derive feature data for a template representative over the set of related cases. In this instance, the features/parameters extracted from the medical images 156A can be compared to the feature data for templates in the medical condition template database 422 to identify a particular medical condition.

In another example, the machine learning algorithm 432 can access the classification database 423, which includes a knowledge base of training data that can be learned from the image database 421 and the template database 422 of previously labeled medical images.

The machine learning algorithm 432 can thus operate according to machine learning tasks as classified into several categories. In supervised learning, the machine learning algorithm 432 builds a mathematical model from a set of data that contains both the inputs and the desired outputs. The set of data is sample data known as “training data”, in order to make predictions or decisions without being explicitly programmed to perform the task. For example, for determining whether the medical image 156 is an abdomen shot, the training data for a supervised learning algorithm would include images with and without example abdomens for specific species, and each image would have a label (the output) designating whether it contained the abdomen. The training data for teaching the machine learning algorithm 132 may be acquired from prior medical image classifications, for example.

In examples, in another category referred to as semi-supervised learning, the machine learning algorithm 432 can develop mathematical models from incomplete training data, where a portion of the sample input does not have labels. In a further aspect, in examples, a classification algorithm can then be used when the outputs are restricted to a limited set of values.

In examples, in another category referred to as unsupervised learning, the machine learning algorithm 432 can build a mathematical model from a set of data that contains only inputs and no desired output labels. In a further aspect, in examples, unsupervised learning algorithms are used to find structure in the x-ray images, such as grouping or clustering of data points. In a further aspect, in examples, unsupervised learning can also discover patterns in the x-ray images, and can group the inputs into categories.

The machine learning algorithm 432 may be executed to identify anatomy in the medical image 156 and then an appropriate label from a labeling scheme can be applied to the medical image 156. The type and amount of anatomy that is possible is a finite number, and thus, the machine learning algorithm 432 may classify the medical images into one of a selected number of groups, such as skull-neck, upper-limb, body-abdomen, lower-limb and other.

Alternative machine learning algorithms 432 may be used to learn and classify the x-ray images, such as deep learning though neural networks or generative models. In examples, deep machine learning may use neural networks to analyze prior medical images through a collection of interconnected processing nodes. In examples, the connections between the nodes may be dynamically weighted. In examples, one or more components of these models (e.g., one or more neural networks) may learn relationships through repeated exposure to data and adjustment of internal weights. In examples, these components (e.g., neural networks) may capture nonlinearity and interactions among independent variables without pre-specification. Whereas traditional regression analysis requires that nonlinearities and interactions be detected and specified manually, neural networks perform the tasks automatically.

In examples, a convolutional neural network is a type of neural network. In examples, layers in a convolutional neural network extract features from the medical image 156. In examples, these deep learning methods learn features that are distinctive for classification. For example, convolution neural networks preserve spatial relationship between pixels of images by learning image features using small squares of input data (e.g., filter kernels for convoluting with an input image may be used). In examples, the convolutional neural network is composed for instance of N convolutional layers, M pooling layers, and at least one fully connected layer.

Still other machine learning algorithms or functions can be implemented to identify anatomy of the x-ray images, such as any number of classifiers that receives input parameters and outputs a classification (e.g., attributes of the image). In examples, support vector machine, Bayesian network, a probabilistic boosting tree, neural network, sparse auto-encoding classifier, or other known or later developed machine learning algorithms may be used. However, many other semi-supervised, supervised, or unsupervised learning may be used. In examples, hierarchal, cascade, or other approaches may be also used.

In another example, image processing includes associating patient identification information with the medical images 156. The computing system 110 can thus access the patient information database 424 to retrieve associated patient identification information. For example, based on the identification of the anatomy in respective medical images 156, the computing system 110 can determine the species of the non-human patient 166, and from the species of the non-human patient 166, the computing system 110 can associate patient identification information with the plurality of medical images 156. The computing system 110 may further utilize timestamps of the medical images 156 cross referenced with scheduling to access the specific patient identification. The patient identification information may include species, breed, age, gender, geographical location, or other information as available from the patient information management system (PIMS) that stores information in the patient information database 424. In some examples, however, it may be the case that the patient information is known and already associated with the medical images 156.

The patient identification information may further be beneficial to assist with the medical image identification. For example, the computing system 110 may be configured to associate patient identification information with the plurality of medical images 156 prior to identification of the medical images, and based on the patient identification information, the computing system 100 further determines the species of the non-human patient 166. Following, the computing system 110 can execute the machine learning algorithm 432 to select a training data set for use by the machine learning algorithm 432 based on the species of the non-human patient 166. The image database 421, the medical condition template database 422, and the classification database 423 all may have different types of training data per different type of species. The patient identification enables the species to be determined and the correct training data set to be used.

The instructions 113 may also include a veterinarian radiology worklist scheme 434. The processor 116 may execute the veterinarian radiology worklist scheme to update the veterinarian radiology worklist 160 based on outputs of the machine learning model 120, as described above.

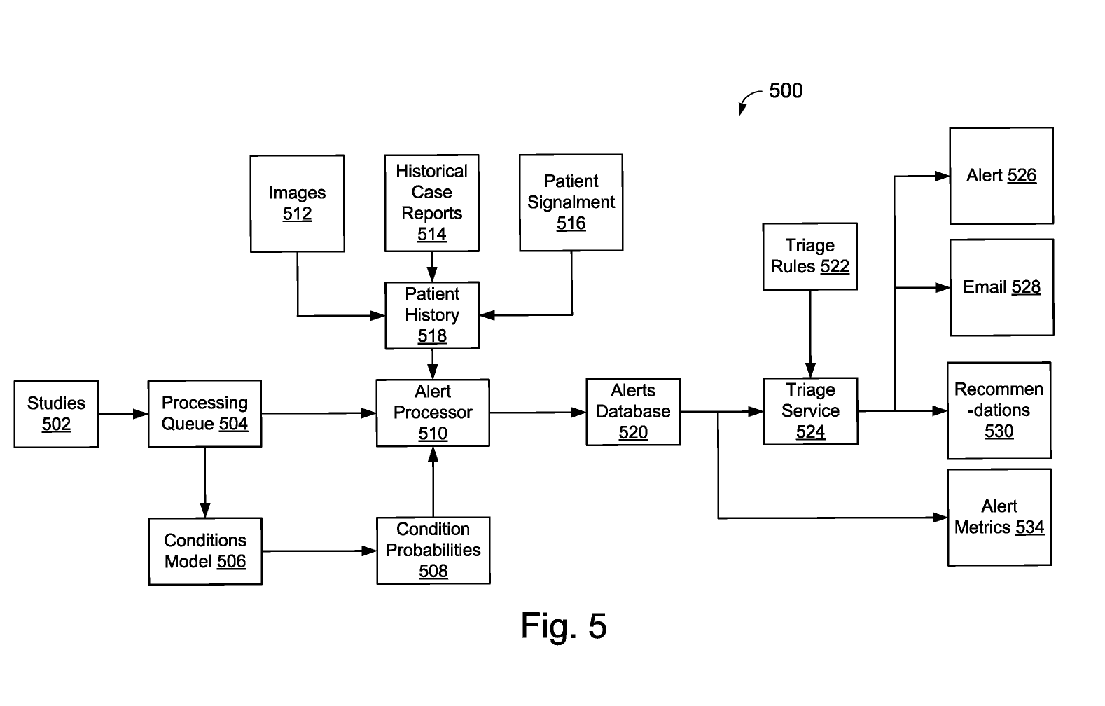

FIG. 5 illustrates an example process 500 for training a machine learning model to update a veterinarian radiology worklist. The process 500 may be used to train the machine learning model 120 of FIG. 1.

According to the process 500, different studies 502 may be provided to a processing queue 504. The studies 502 may be provided a plurality of different users (e.g., medical professionals) that relate certain images to certain medical conditions. The processing queue 504 may provide the studies to an alert processor 510 and to a conditions model 506. The condition model 506 may determine condition probabilities 508 based on the studies 502. For example, condition probabilities 508 may indicate a probability that a medical condition exist based on symptoms identified from the studies 502. The condition probabilities 508 may also be provided to the alert processor 510.

The alert processor 510 may also receive patient history 518. The patient history 518 may include images 512 (e.g., radiology images) from different patients, historical case reports 514 used to diagnose medical conditions, and patient signalment 516 information. Based on the studies 502, the condition probabilities 508, and the patient history 518, the alert processor 510 may generate an alert that is provided to an alerts database 520. The alert may indicate a presence of a medical condition.

Alerts in the alert database 520 may be provided to a triage service 524 that is governed by triage rules 522 and metrics 534 associated with the alerts may be output. The triage service 524 may output an alert 526 indicative of the medical condition, an email 528 indicative of the medical condition, and/or recommendations 530 for the medical condition.

### EXAMPLE METHODS AND ASPECTS

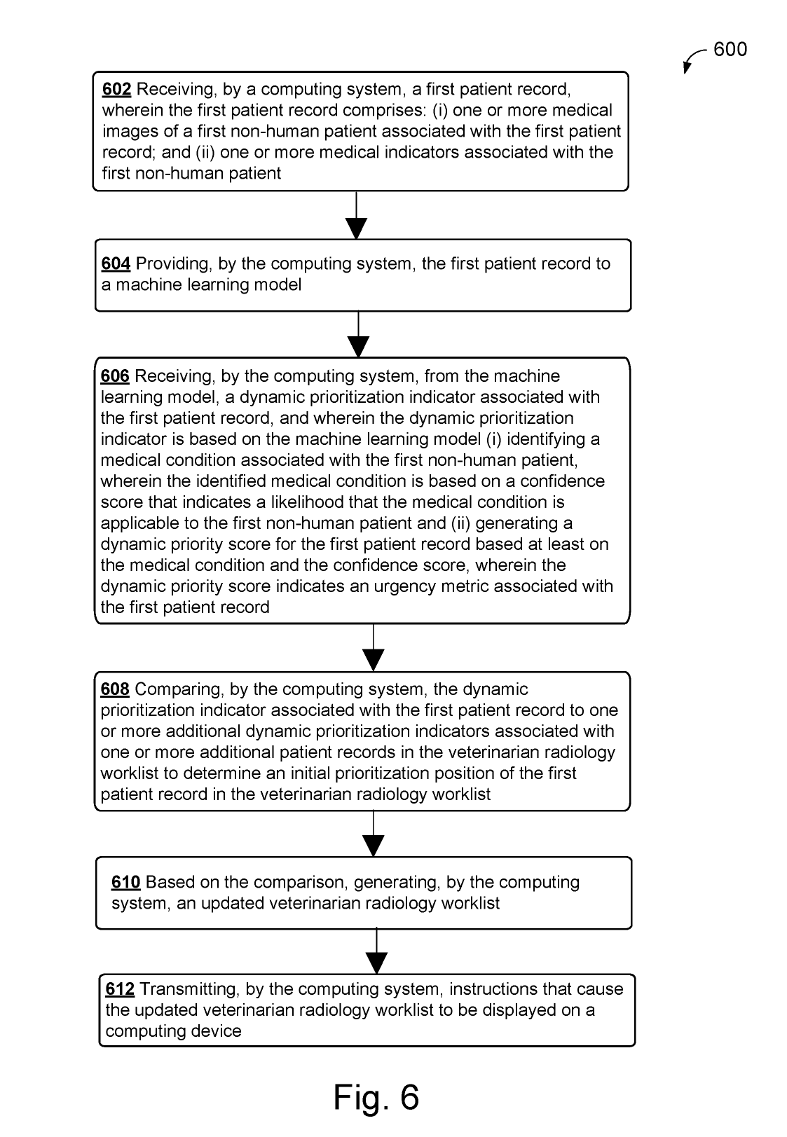

Now referring to FIG. 6, an example method of updating a veterinary radiology worklist is disclosed.

The method 600 shown in FIG. 6 presents an example of a method that could be used with the components shown in FIGS. 1-5, for example. In other examples, components of the devices and/or systems may be arranged to be adapted to, capable of, or suited for performing the functions, such as when operated in a specific manner. The method 600 may include one or more operations, functions, or actions as illustrated by one or more of blocks 602-612. Although the blocks are illustrated in a sequential order, these blocks may also be performed in parallel, and/or in a different order than those described herein.

Also, the various blocks may be combined into fewer blocks, divided into additional blocks, and/or removed based upon the desired implementation.

At block 602, the method 600 includes receiving, by a computing system, a first patient record. The first patient record includes (i) one or more medical images of a first non-human patient associated with the first patient record and (ii) one or more medical indicators associated with the first non-human patient.

At block 604, the method 600 includes providing, by the computing system, the first patient record to a machine learning model.

At block 606, the method 600 includes receiving, by the computing system, from the machine learning model, a dynamic prioritization indicator associated with the first patient record. The dynamic prioritization indicator is based on the machine learning model identifying a medical condition associated with the first non-human patient. The identified medical condition is based on a confidence score that indicates a likelihood that the medical condition is applicable to the first non-human patient. The dynamic prioritization indicator is also based on the machine learning model generating a dynamic priority score for the first patient record based at least on the medical condition and the confidence score. The dynamic priority score indicates an urgency metric associated with the first patient record.

At block 608, the method 600 includes comparing, by the computing system, the dynamic prioritization indicator associated with the first patient record to one or more additional dynamic prioritization indicators associated with one or more additional patient records in the veterinarian radiology worklist to determine an initial prioritization position of the first patient record in the veterinarian radiology worklist.

At block 610, the method 600 includes, based on the comparison, generating, by the computing system, an updated veterinarian radiology worklist.

At block 612, the method 600 includes transmitting, by the computing system, instructions that cause the updated veterinarian radiology worklist to be displayed on a computing device.

In some examples of the method 600, wherein the one or more medical indicators associated with the first non-human patient comprises information stored in association with one or more medical records associated with the first non-human patient.

In some examples of the method 600, the one or more medical indicators associated with the first non-human patient comprise one or more of the following: (i) an age of the first non-human patient; (ii) a weight of the first non-human patient; (iii) a species of the first non-human patient; (iv) a breed of the first non-human patient; (v) one or more symptoms of the first non-human patient; (vi) a preexisting condition of the first non-human patient; and (vii) a geographical location of the first non-human patient.

In some examples of the method 600, the one or more medical indicators associated with the first non-human patient comprises information stored in association with one or more medical records associated with a second non-human patient that shares one or more attributes with the first non-human patient. The one or more attributes comprises one or more of the following: (i) age; (ii) weight; (iii) species; (iv) breed; (v) one or more symptoms; (vi) a preexisting condition; and (vii) a geographical location.

In some examples, the method 600 may include generating, by the computing system, a reprioritized updated veterinarian radiology worklist. The method 600 may also include transmitting, by the computing system, instructions that cause the reprioritized updated veterinarian radiological worklist to be displayed on the computing device.

In some examples of the method 600, generating the reprioritized updated veterinarian radiology worklist comprises determining, by the computing system, a threshold elapsed time period for reprioritizing the first patient record in the updated veterinarian radiology worklist based on the urgency metric associated with the first patient record. Generating the reprioritized updated veterinarian radiology worklist also comprises monitoring, by the computing system, an elapsed time since generating the updated veterinarian radiology worklist. Generating the reprioritized updated veterinarian radiology worklist also comprises determining, by the computing system, whether an action has been performed on the first patient record prior to the elapsed time exceeding the threshold elapsed time period.

In some examples, in response to determining that no action has been performed on the first patient record prior to the elapsed time exceeding the threshold elapsed time period, the method 600 includes updating the dynamic priority score for the first patient record based on an escalated urgency metric. The method 600 may also include generating an updated dynamic prioritization indicator associated with the first patient record based on the updated dynamic priority score for the first patient record. The method 600 may also include comparing the updated dynamic prioritization indicator associated with the first patient record to the one or more additional dynamic prioritization indicators associated with the one or more additional patient records to determine an updated prioritization position of the first patient record. The method 600 may also include reprioritizing, by the computing system, the first patient record in the updated veterinarian radiology worklist according to the updated prioritization position.

In some examples of the method 600, the updated prioritization position has a higher priority than the initial prioritization position.

In some examples, in response to determining that no action has been performed on the first patient record prior to the elapsed time exceeding the threshold elapsed time period, the method 600 further includes transmitting, by the computing system, instructions that cause the updated radiology worklist to be displayed on a second computing device.

In some examples, the computing device is a first computing device associated with a first radiologist, and the second computing device is associated with a second radiologist.

In some examples, the method 600 also includes classifying the first patient record based on the dynamic priority score. A classification of the first patient record is included in the updated veterinarian radiology worklist.

In some examples of the method 600, the first patient record is classified as a critical case if action should be performed on the first patient record before expiration of a first time period, the first patient record is classified as a priority case if action should be performed on the first patient record before expiration of a second time period, and the first patient record is classified as a routine case if action should be performed on the first patient record before expiration of a third time period.

In some examples of the method 600, the first time period is one hour, the second time period is four hours, and the third time period is twenty-four hours.

In some examples of the method 600, the classification of the first patient record is configured to change based on an elapsed time since inserting the first patient record in the updated veterinarian radiology worklist.

In some examples of the method 600, determination of the dynamic priority score by the machine learning model is further based on a medical provider associated with the first non-human patient.

In some examples of the method 600, the one or more medical images comprise radiology images of the first non-human patient.

In some examples, prior to receiving the first patient record, the method includes training the machine learning model. Training the machine learning model may include identifying the medical condition and identifying whether an action has been performed on a plurality of patient records associated with a plurality of non-human patients that share one or more attributes with the first non-human patient prior to an elapsed time exceeding a threshold elapsed time period. Training the machine learning model may also include receiving an indication of an efficacy of treating the medical condition in the plurality of non-human patient. The method 600 may also include receiving an updated indication of whether the action has been performed on the first patient record prior to the elapsed time exceeding the threshold elapsed time period. The method 600 may also include retraining the machine learning model based on the updated indication.

It should now be understood that embodiments according to the present disclosure are directed to systems and methods for dynamically prioritizing veterinary radiology worklists. As outlined above, by dynamically prioritizing veterinary radiology worklists, radiological assessments can be performed in a manner ordered by medical importance, ensuring that critical patients are prioritized. Without being bound by theory, veterinary patients (e.g., non-human animals) present challenges that are not found in the human health space. For example and in particular, non-human animals are unable to communicate verbally with health care providers and are thus unable to express their medical condition (e.g., what symptoms they are experiencing, where pain exists, how long symptoms have been present, etc.). By contrast human patients are generally able to verbalize their medical condition, symptoms, and the like. As such, radiological consults in the human space can be prioritized based on information gathered directly from the patient. Because non-human patents cannot verbalize their condition, information relevant to prioritization is unavailable, and alternate systems and methods that prioritize the radiological worklist based at least in part on medical images and medical indicators are necessary. Moreover, because embodiments according to the present disclosure dynamically prioritize radiological worklists based at least in part on digital medical images and medical indicators, the methods according to the present disclosure cannot practically be performed in the human mind.

The singular forms of the articles “a,” “an,” and “the” include plural references unless the context clearly indicates otherwise. For example, the term "a compound" or "at least one compound" can include a plurality of compounds, including mixtures thereof.

Various aspects and embodiments have been disclosed herein, but other aspects and embodiments will certainly be apparent to those skilled in the art. Additionally, the various aspects and embodiments disclosed herein are provided for explanatory purposes and are not intended to be limiting, with the true scope being indicated by the following claims.

What is claimed is:

1. A computer-implemented method for updating a veterinarian radiology worklist, the computer-implemented method comprising:
receiving, by a computing system, a first patient record, wherein the first patient record comprises: (i) one or more medical images of a first non-human patient associated with the first patient record; and (ii) one or more medical indicators associated with the first non-human patient;
providing, by the computing system, the first patient record to a machine learning model;
receiving, by the computing system, from the machine learning model, a dynamic prioritization indicator associated with the first patient record, and wherein the dynamic prioritization indicator is based on the machine learning model:
identifying a medical condition associated with the first non-human patient, wherein the identified medical condition is based on a confidence score that indicates a likelihood that the medical condition is applicable to the first non-human patient; and
generating a dynamic priority score for the first patient record based at least on the medical condition and the confidence score, wherein the dynamic priority score indicates an urgency metric associated with the first patient record;
comparing, by the computing system, the dynamic prioritization indicator associated with the first patient record to one or more additional dynamic prioritization indicators associated with one or more additional patient records in the veterinarian radiology worklist to determine an initial prioritization position of the first patient record in the veterinarian radiology worklist;
based on the comparison, generating, by the computing system, an updated veterinarian radiology worklist; and
transmitting, by the computing system, instructions that cause the updated veterinarian radiology worklist to be displayed on a computing device.

2. The computer-implemented method of claim 1, wherein the one or more medical indicators associated with the first non-human patient comprises information stored in association with one or more medical records associated with the first non-human patient.

3. The computer-implemented method of claim 1, wherein the one or more medical indicators associated with the first non-human patient comprise one or more of the following: (i) an age of the first non-human patient; (ii) a weight of the first non-human patient; (iii) a species of the first non-human patient; (iv) a breed of the first non-human patient; (v) one or more symptoms of the first non-human patient; (vi) a preexisting condition of the first non-human patient; and (vii) a geographical location of the first non-human patient.

4. The computer-implemented method of claim 1, wherein the one or more medical indicators associated with the first non-human patient comprises information stored in association with one or more medical records associated with a second non-human patient that shares one or more attributes with the first non-human patient.

5. The computer-implemented method of claim 4, wherein the one or more attributes comprises one or more of the following: (i) age; (ii) weight; (iii) species; (iv) breed; (v) one or more symptoms; (vi) a preexisting condition; and (vii) a geographical location.

6. The computer-implemented method of claim 1, further comprising:
generating, by the computing system, a reprioritized updated veterinarian radiology worklist; and
transmitting, by the computing system, instructions that cause the reprioritized updated veterinarian radiology worklist to be displayed on the computing device.

7. The computer-implemented method of claim 6, wherein generating the reprioritized updated veterinarian radiology worklist comprises:
determining, by the computing system, a threshold elapsed time period for reprioritizing the first patient record in the updated veterinarian radiology worklist based on the urgency metric associated with the first patient record;
monitoring, by the computing system, an elapsed time since generating the updated veterinarian radiology worklist; and
determining, by the computing system, whether an action has been performed on the first patient record prior to the elapsed time exceeding the threshold elapsed time period.

8. The method of claim 7, further comprising, in response to determining that no action has been performed on the first patient record prior to the elapsed time exceeding the threshold elapsed time period:
updating the dynamic priority score for the first patient record based on an escalated urgency metric;
generating an updated dynamic prioritization indicator associated with the first patient record based on the updated dynamic priority score for the first patient record;
comparing the updated dynamic prioritization indicator associated with the first patient record to the one or more additional dynamic prioritization indicators associated with the one or more additional patient records to determine an updated prioritization position of the first patient record; and
reprioritizing, by the computing system, the first patient record in the updated veterinarian radiology worklist according to the updated prioritization position.

9. The method of claim 8, wherein the updated prioritization position has a higher priority than the initial prioritization position.

10. The computer-implemented method of claim 7, in response to determining that no action has been performed on the first patient record prior to the elapsed time exceeding the threshold elapsed time period, further comprising transmitting, by the computing system, instructions that cause the updated radiology worklist to be displayed on a second computing device.

11. The computer-implemented method of claim 10, wherein the computing device is a first computing device associated with a first radiologist, and wherein the second computing device is associated with a second radiologist.

12. The computer-implemented method of claim 1, further comprising classifying the first patient record based on the dynamic priority score, wherein a classification of the first patient record is included in the updated veterinarian radiology worklist.

13. The computer-implemented method of claim 12, wherein the first patient record is classified as a critical case if action should be performed on the first patient record before expiration of a first time period, wherein the first patient record is classified as a priority case if action should be performed on the first patient record before expiration of a second time period, and wherein the first patient record is classified as a routine case if action should be performed on the first patient record before expiration of a third time period.

14. The computer-implemented method of claim 13, wherein the first time period is one hour, wherein the second time period is four hours, and wherein the third time period is twenty-four hours.

15. The computer-implemented method of claim 12, wherein the classification of the first patient record is configured to change based on an elapsed time since inserting the first patient record in the updated veterinarian radiology worklist.

16. The computer-implemented method of claim 1, wherein determination of the dynamic priority score by the machine learning model is further based on a medical provider associated with the first non-human patient.

17. The computer-implemented method of claim 1, wherein the one or more medical images comprise radiology images of the first non-human patient.

18. The computer-implemented method of claim 1, further comprising:
prior to receiving the first patient record, training the machine learning model, wherein training the machine learning model comprises:
identifying the medical condition;
identifying whether an action has been performed on a plurality of patient records associated with a plurality of non-human patients that share one or more attributes with the first non-human patient prior to an elapsed time exceeding a threshold elapsed time period; and
receiving an indication of an efficacy of treating the medical condition in the plurality of non-human patient;
receiving an updated indication of whether the action has been performed on the first patient record prior to the elapsed time exceeding the threshold elapsed time period; and
retraining the machine learning model based on the updated indication.

19. A computing device comprising:
one or more processors; and
a tangible, non-transitory computer-readable medium comprising instructions that, when executed by the one or more processors, cause the one or more processors to perform operations comprising:
receiving a first patient record, wherein the first patient record comprises: (i) one or more medical images of a first non-human patient associated with the first patient record; and (ii) one or more medical indicators associated with the first non-human patient;
providing the first patient record to a machine learning model;
receiving a dynamic prioritization indicator associated with the first patient record, and wherein the dynamic prioritization indicator is based on the machine learning model:
identifying a medical condition associated with the first non-human patient, wherein the identified medical condition is based on a confidence score that indicates a likelihood that the medical condition is applicable to the first non-human patient; and
generating a dynamic priority score for the first patient record based at least on the medical condition and the confidence score, wherein the dynamic priority score indicates an urgency metric associated with the first patient record;
comparing the dynamic prioritization indicator associated with the first patient record to one or more additional dynamic prioritization indicators associated with one or more additional patient records in a veterinarian radiology worklist to determine an initial prioritization position of the first patient record in the veterinarian radiology worklist;
based on the comparison, generating an updated veterinarian radiology worklist; and
transmitting instructions that cause an updated veterinarian radiology worklist to be displayed on a computing device.

20. A tangible, non-transitory computer-readable medium comprising instructions that, when executed by one or more processors, cause the one or more processors to perform operations comprising:
receiving a first patient record, wherein the first patient record comprises: (i) one or more medical images of a first non-human patient associated with the first patient record; and (ii) one or more medical indicators associated with the first non-human patient;
providing the first patient record to a machine learning model;
receiving a dynamic prioritization indicator associated with the first patient record, and wherein the dynamic prioritization indicator is based on the machine learning model:
identifying a medical condition associated with the first non-human patient, wherein the identified medical condition is based on a confidence score that indicates a likelihood that the medical condition is applicable to the first non-human patient; and
generating a dynamic priority score for the first patient record based at least on the medical condition and the confidence score, wherein the dynamic priority score indicates an urgency metric associated with the first patient record;
comparing the dynamic prioritization indicator associated with the first patient record to one or more additional dynamic prioritization indicators associated with one or more additional patient records in a veterinarian radiology worklist to determine an initial prioritization position of the first patient record in the veterinarian radiology worklist;
based on the comparison, generating an updated veterinarian radiology worklist; and
transmitting instructions that cause an updated veterinarian radiology worklist to be displayed on a computing device.
